(19) United States
(12) Patent Application Publication
(10) Pub. No.: US 2001/0005785 A1
(43) Pub. Date: Jun. 28, 2001

(54) Probe for the small intestines

(76) Inventor: Hans Sachse, Nurnberg (DE)

Correspondence Address: Herbret B. Keil, KEIL & WEINKAUF, 1101 Connecticut Ave., N.W., Washington, DC, 20036, US

(21) Appl. No.: 09/742,354

(22) Filed: Dec. 22, 2000

(30) Foreign Application Priority Data
Dec. 22, 1999 (DE) P 199 62 473.9
Aug. 29, 2000 (DE) P 100 42 330.2

(51) Int. Cl. A61M 25/00

(52) U.S. Cl. 604/530; 604/523

(57) ABSTRACT

The inventive probe has a probe tube and a guide stylet. The probe tube has a flexible, easily deformable wall and a tip which produces an opaque shadow, with one or several conduits for the flow-through of the X-ray contrast medium. The probe tube is introduced either through the esophagus into the stomach. Once the tip has arrived there, the physician attempts to find the pylorus with the probe tip by means of X-ray control and insert the probe therein. The invention features an elastic-walled probe tube with a soft tip area, such that it substantially adapts itself to the curvature of the tip area of the guide stylet and is not capable of counteracting the curvature of the tip area or straightening it. The tip of this guide stylet is sufficiently rigid that, when inserted into the probe tube as far as the tip of the latter, it forces its own shape on the probe tip, which makes it easier for the physician to find the way into and through the pylorus with this curved tip.

### BACKGROUND OF THE INVENTION

Various medical conditions require introduction of a probe into the small intestines for treatment or diagnosis. For example, such a probe can be used to introduce nutrient fluids or to deliver X-ray contrast media, thereby facilitating the representation of defined intestinal sections. It is desirable to make the passage of the probe through the pylorus into the small intestines easier by making the probe tip steerable. This aim is pursued in German patent applications DE 43 20 186 and DE 43 33 090 which are hereby incorporated by reference. Because the probe must make a loop in the stomach during this process in order to find the pylorus located close to the diaphragm, a steerable probe tip makes this process easier and less time consuming. German patent application DE 43 20 186 describes a double stylet used for steering or deforming the probe tip in the stomach. German patent application DE 43 33 090 describes a probe wall having a memory effect in the tip area, i.e. a predetermined curvature, which is counteracted by a stiff guide stylet for the introduction process. In another variation, DE 43 33 090 describes a single stylet with a curved tip with a memory effect which is counteracted by the stiff wall of the probe for the introduction process. Once in the stomach, the curvature of the tip of the guide stylet is released by pushing the guide stylet out of the tip of the probe.

### SUMMARY OF THE INVENTION

It is an object of the present invention to provide a cost-effective probe for the small intestines, whose tip is steerable and which does not overly stress the patient during use, and saves the physician time.

The present invention has a probe tube and a guide stylet. The probe tube has a flexible, easily deformable wall and preferably a tip which produces an opaque shadow, mostly in the shape of a metal button with one or several conduits for the flow-through of the X-ray contrast medium. The probe tube is introduced either through the nasopharyngeal cavity or the oral cavity and the esophagus into the stomach. Once the tip has arrived there, the physician attempts to find the pylorus with the probe tip by means of X-ray control and insert the probe therein.

The invention features an elastic-walled probe tube with a soft tip area, such that it substantially adapts itself to the curvature of the tip area of the guide stylet and is not capable of counteracting the curvature of the tip area or straightening it. Moreover, the length of the guide stylet is preferably such that the tip of the guide stylet cannot leave the tip of the probe tube. Once the tip of the probe tube without the guide stylet inside it has reached the center of the stomach, the guide stylet is introduced into the horizontal probe tube from outside the patient in a second step. This guide stylet has a curved tip and is preferably made of metal, for example nitinol, with a memory effect. In this case the tip of this guide stylet is so solid that, when inserted into the probe tube as far as the tip of the latter, it forces its own shape on the probe tip, which makes it easier for the physician to find the way into and through the pylorus with this curved tip.

### BRIEF DESCRIPTION OF THE DRAWINGS

FIG. 1 depicts both the probe tube and the guide stylet.

FIG. 2 depicts a probe with both an inner and an outer probe tube.

FIG. 3 depicts a probe tube with a sheath.

### DETAILED DESCRIPTION OF THE INVENTION

The flexibility of the wall of the probe tube and the flexibility of the guide stylet are preferably matched to each other such that the guide stylet only slightly deforms the probe tube which is conducted in a straight line through the esophagus and, when reaching the wide stomach lumen, it forces its curvature on the area of the probe tip which is not guided here, so that now the probe tube with the inserted guide stylet constitutes a unit with a curved tip. It is possible to screw the end of the probe tube and the guide stylet together for further manipulations.

The patient will notice the passage of the guide stylet through the probe tube lying in the esophagus. However, the patient will tolerate this process relatively well while the probe tube wall rests immovably against the wall of the esophagus, and will not experience pain. In other words, the esophagus provides additional support and rigidity to the probe tube wall preventing the guide stylet from imparting its shape on the probe tube in the esophagus. The wide stomach lumen does not provide such support.

In a preferred embodiment, the guide stylet has a spherical tip. The spherical shape prevents both snags on the inner wall of the probe tube when the stylet is pushed forward and also prevents penetration of the tip area of the probe tube which could cause injury to the patient.

In another preferred embodiment, the wall of the probe tube is more flexible in the tip area relative to the rest of the wall of the probe tube in order to make possible a better adaptation to the shape of the guide stylet. This may be achieved by reducing the thickness of the wall of the probe tube in the tip area, by making the tip area from a different material than the rest of the probe tube or by providing a sheath on the outside of the probe tube. Alternatively, and preferably, this effect is achieved by introducing a second inner stiffening probe tube. The stiffening probe tube may be displaceable with respect to the first probe tube or fixed thereto.

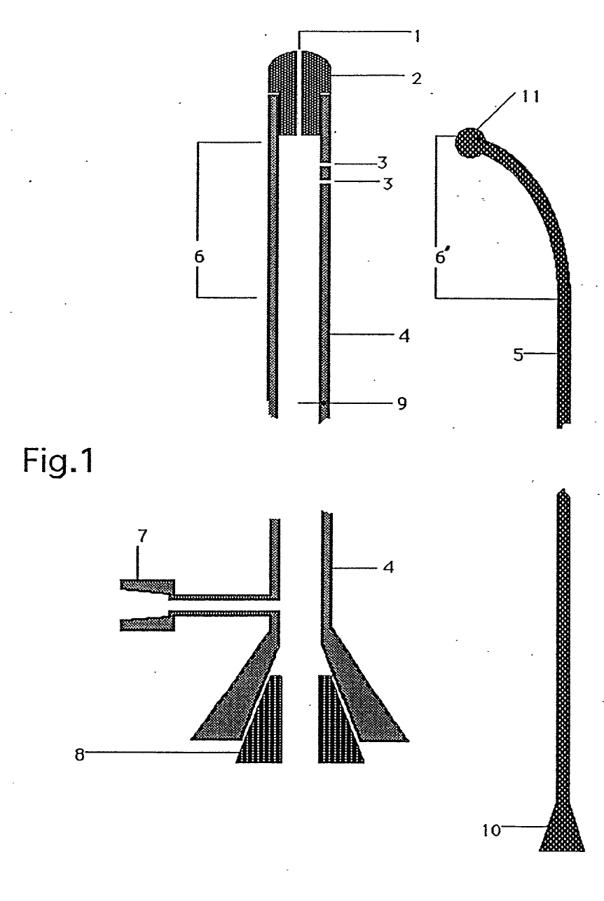

With reference to FIG. 1, the tip of the probe tube preferably has a metal button (2) with a central bore (1). The metal button (2) lets the physician see the exact position of the tip of the probe tube in the X-ray image. The perforation (1), as well as the outlet openings (3), permit the exit of fluid from the probe tube lumen (9). The wall of the probe tube (4), preferably made of a plastic material, is flexible enough that it can be deformed by the inserted guide stylet, but otherwise is sufficiently stiff for being introduced through the esophagus into the stomach. The probe tube further has a tip area (6) which is preferably more flexible than the rest of the probe tube. The guide stylet (5) is curved in the tip area (6′) and has a handle (10). Its material is elastic and returns to its predetermined shape when not under stress. The spray connector (7) permits the continuous administration of contrast medium during manipulation. A valve-like termination (8) permits the fixation of the position of the guide stylet in the probe tube, or the screwing of the two together. The probe tube has a lumen (9). The guide stylet has a handle for the improved rotation of the probe tube when the guide stylet is inserted into it. The guide stylet may have a straight tip (12) or preferably a spherical tip (11) to permit improved sliding capability of the tip of the guide stylet in the lumen of the probe tube and also prevent the guide stylet from leaving the tip of the probe tube through perforation (1) or the outlet openings (3) which could cause injuries to the patient.

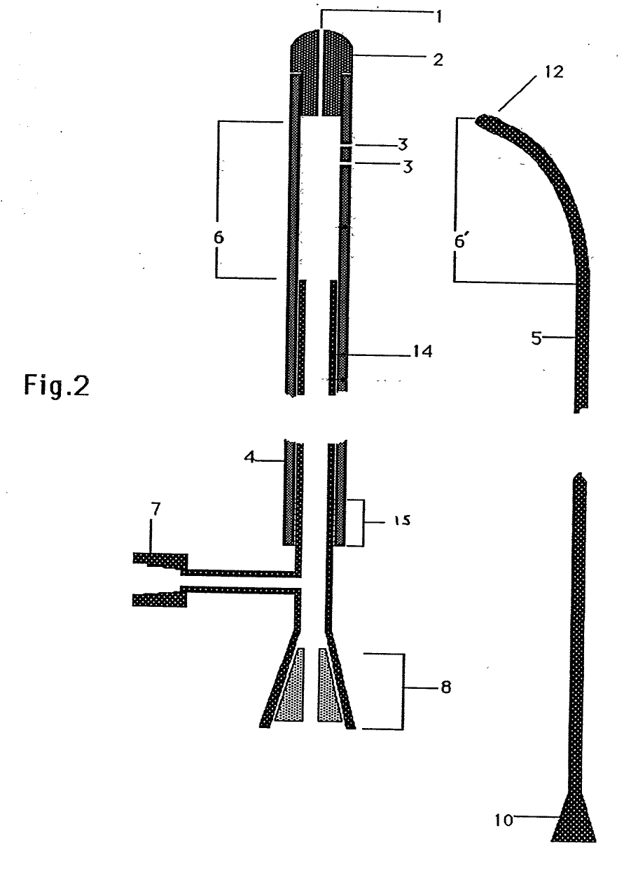

FIG. 2 shows the preferred embodiment with an outer probe tube (4) and an inner stiffening tube (14). The wall of the probe tube (4) is so flexible, preferably made of a plastic material, that it can be deformed by the inserted guide stylet (5), which is bent in the tip area. The inner tube (14), which in the present exemplary embodiment is fixedly connected to the probe tube (4) in the retaining area (15), has been inserted into the flexible probe tube (4) for stiffening the probe tube, except in the tip area. In this embodiment, the spray connector (7) and termination (8) are on to the inner tube (14) rather than the probe tube (4).

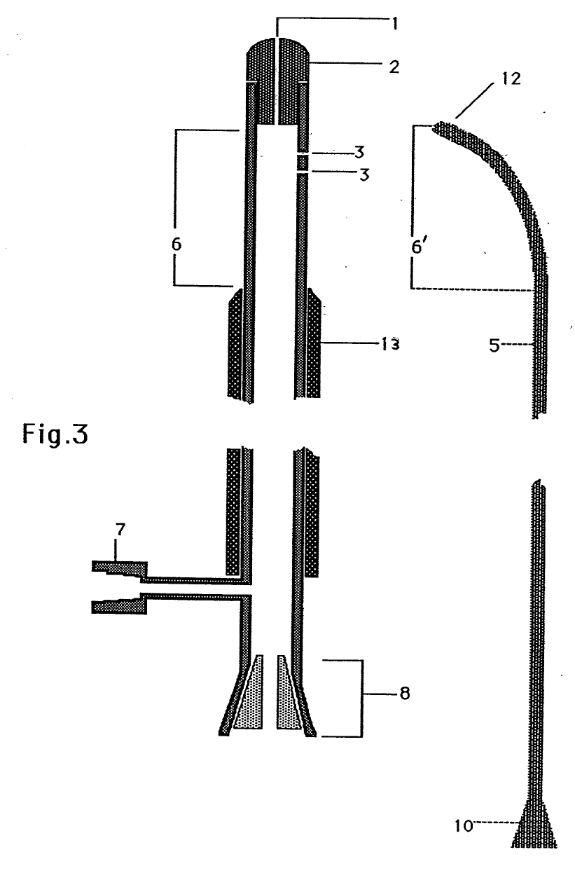

FIG. 3 shows the probe tube (4) enclosed by an outer stiffening tube or sheath (13).

The invention also relates to a process for delivering fluid containing, e.g., nutrients, X-ray medium or medication, to the small intestine. As described above, the physician can insert the inventive probe tube into the stomach by way of the esophagus. It is also possible to use a second strait stylet during the introduction of the probe into the esophagus and stomach to provide it with increased stiffness during the introduction process. The physician can then insert the guide stylet into the probe tube such that the guide stylet imparts its curvature on the probe tube, thus facilitating insertion into the pylorus and into the small intestines. Preferably, insertion into the pylorus is further aided by first spraying X-ray contrast medium into the probe tube from a lateral connection of the probe tube, which then exits the tip of the probe tube. The physician can then better visualize what he is doing with the aid of an X-ray device. When the tip of the probe tube has reached its end position in the small intestine, the guide stylet can be pulled out and, with the lumen of the probe tube now completely empty, further X-ray contrast medium or nutrient fluids can be introduced into the small intestine.

1. A probe for the small intestines comprising
a probe tube (4) comprising a tip area (6) and a tip (2) having an outlet opening (1), and
a guide stylet (5) with a curved tip area (6′),
wherein the flexibility of the probe tube (4) and the guide stylet (5) are such that, in the absence of an external force, the shape of the guide stylet (5) is substantially imparted on the tip area (6) of the probe tube (4) when inserted therein.

2. The probe of claim 1, wherein the probe tube (4) is more flexible in the tip area (6) than in the rest of the tube.

3. The probe of claim 1, wherein the guide stylet has a spherically shaped tip (11).

4. The probe of claim 1, wherein the probe tube is thinner in the tip area (6) than in the rest of the probe tube (4).

5. The probe of claim 1, further comprising additional outlet openings (3) in the tip area (6).

6. The probe of claim 1, wherein the probe tube (4) is made from a plastic material.

7. The probe of claim 1, wherein the guide stylet (5) is made from a metal having a memory-effect.

8. The probe of claim 1, further comprising a termination (8) for connecting the guide stylet (5) to the probe tube (4).

9. The probe tube of claim 1, further comprising a spray connector (7) on the probe tube (4).

10. The probe of claim 1, wherein the guide stylet (5) is sufficiently flexible so that it does not impart its shape on the probe tube (4) when the probe tube (4) is inserted in a human esophagus.

11. The probe of claim 1, further comprising and an inner stiffening tube (14) which does not extend into the tip area (6) of the probe tube (4).

12. The probe of claim 11, wherein the inner stiffening tube (14) is sufficiently rigid so that when the inner stiffening tube (14) and the guide stylet (5) are inserted into the probe tube (4), the guide stylet (5) does not substantially impart its shape on the inner stiffening tube (14).

13. The probe of claim 1, further comprising a sheath (13) on the probe tube (4) which is sufficiently rigid so that when the guide stylet (5) is inserted into the probe tube (4), the guide stylet (5) does not substantially impart its shape on the probe tube (4).

14. A process for delivering fluid to the small intestine with the probe of claim 1, comprising the steps of
inserting the probe tube (4) into a patient's stomach by way of the patient's esophagus,
inserting the guide stylet (5) into the probe tube (4),
inserting the tip (2) of the probe tube (4) into the patient's small intestines by way of the patient's pylorus, and
passing the fluid through a lumen (9) and the opening (1) of the probe tube.

15. The process of claim 14, further comprising the step of spraying X-ray contrast medium into the probe tube (4) prior to inserting the tip (2) of the probe tube (4) into the small intestines.

16. The process of claim 14, further comprising inserting a second straight stylet into the probe tube (4) prior to inserting the probe tube (4) into the stomach.
